(19) United States
(12) Patent Application Publication
(10) Pub. No.: US 2015/0306038 A1
(43) Pub. Date: Oct. 29, 2015

(54) A MICROPARTICLE COMPOSITION COMPRISING A PROBIOTIC, CROSS-LINKABLE REAGENT AND AN EMULSION CONTAINING A HYDROPHOBIC ACTIVE

(71) Applicant: ProGel Pty Ltd, Brisbane (AU)

(72) Inventors: Lai Tran, Carina Heights (AU); Su Hung Ching, Banyo (AU); Cameron Turner, Rochedale South (AU)

(73) Assignee: ProGel Pty Ltd, Brisbane (AU)

(21) Appl. No.: 14/648,310

(22) PCT Filed: Nov. 29, 2013
(86) PCT No.: PCT/AU2013/001384

§ 371 Date: May 29, 2015

(30) Foreign Application Priority Data
Nov. 29, 2012 (AU) 2012905215

(51) Int. Cl. A61K 9/16; A61K 35/745; A61K 35/60; A61K 35/747

(52) U.S. Cl. CPC A61K 9/16; A61K 35/747; A61K 35/745; A61K 9/1652; A61K 35/60; A61K 9/1694

(57) ABSTRACT

The present invention relates to microparticles, methods of producing microparticles and microparticle precursor compositions. In particular, it relates to a method of producing a microparticle precursor composition comprising blending an emulsion comprising a hydrophobic active with a probiotic to form a probiotic-containing emulsion; and blending the probiotic-containing emulsion with a cross-linkable reagent.

### TECHNICAL FIELD

The present invention relates to microparticles, methods of producing microparticles and microparticle precursor compositions.

### BACKGROUND

In a healthy gut, there is a balance between beneficial and pathogenic bacteria. Various factors including food, stress, illness or infection and medications can disrupt this balance leading to an excess of pathogenic bacteria. This imbalance may lead to bloating, gas and constipation. Over recent years, there has been a significant increase in the use of probiotic micro-organisms (hereinafter “probiotics”) to address this imbalance. It is believed that probiotics can inhibit or influence the growth and/or metabolism of pathogenic bacteria in the intestinal tract. Probiotics may also activate immune function. For this reason, there is considerable interest in incorporating probiotics into nutritional supplements or foodstuffs.

There are difficulties associated with incorporating probiotics into nutritional supplements or foodstuffs. One primary difficulty is having or maintaining an adequate number of viable micro-organisms in the relevant product. If the concentration of the viable probiotics in the food product does not exceed a certain threshold value; the beneficial effect of the probiotics is not provided. Temperature and exposure to oxygen, water and acids can affect probiotic viability. Furthermore, the shear forces, generated in certain production processes such as high-speed blending, emulsification and homogenization may result in cell disruption and losses in viability.

It can be desirable to combine a probiotic with one or more other actives to produce a microparticle with added beneficial activity. However, when preparing a microparticle precursor composition, it can be difficult to, combine the probiotic with other actives due to the sensitivity of the probiotic. Blending any other actives with the remaining components of the microparticle precursor composition, including the probiotic, can involve shear rates that disrupt the cells of the probiotic and compromise its viability.

An opportunity therefore remains to address or ameliorate one or more shortcomings or disadvantages associated with existing methods of combining probiotics with other actives and/or to at least provide a useful alternative thereto.

### SUMMARY OF THE INVENTION

The present invention provides a microparticle comprising a hydrophobic active and a probiotic distributed within a cross-linked matrix. This aspect of the invention also provides a microparticle precursor composition comprising a blend of a probiotic, a cross-linkable reagent and an emulsion comprising a hydrophobic active.

The invention further provides a method of producing a microparticle precursor composition comprising blending an emulsion comprising a hydrophobic active with a probiotic to form a probiotic-containing emulsion; and blending the probiotic-containing emulsion with a cross-linkable reagent. Both blending steps will be performed at suitably low shear rates to ensure that probiotic viability is not unduly compromised.

The present invention also provides a method of producing microparticles comprising providing a microparticle precursor composition in accordance with the present invention in a finely divided state; and exposing the finely divided microparticle precursor composition to a cross-linking reagent that reacts with the cross-linkable reagent of the microparticle precursor composition to form microparticles.

These and other aspects of the invention, including a product comprising microparticles according to the present invention are described in more detail below.

### DETAILED DESCRIPTION

In the present invention, by providing the hydrophobic active in an emulsion, the hydrophobic active may be readily blended with the probiotic and other components of the microparticle precursor composition. More particularly, blending may be undertaken without subjecting the probiotic to shear forces that may compromise probiotic viability by causing disruption of the probiotic cells. Thus, the present invention may improve probiotic survival during microparticle production. Through improved probiotic survival, microparticles with beneficial levels of probiotic can be more readily produced. As a result, in one or more embodiments it may be possible to reduce the amount of probiotic used when compared with conventional methodologies that involve high shear processing. In such approaches the amount of probiotic used is increased to compensate for losses that would occur during microparticle production. As the present invention enables microparticles to be produced by methodologies in which the probiotic is not subjected to high shear, it is possible to reduce the amount of probiotic that is used.

In the microparticle precursor composition of the present invention, the hydrophobic active is present in an emulsion and the probiotic is believed to be dispersed within the emulsion. More particularly, without being bound by theory, it is believe that probiotic cells may be located within the interstitial spaces provided between droplets of the emulsion. This structural feature is believed to be retained in microparticles formed from the precursor composition. The presence of cells within interstitial spaces between droplets is believed to provide some form of protection (shielding) to the probiotic cells so that probiotic survival may be improved. Thus, it has been found that by suitable mixing of a probiotic with an emulsion a beneficial effect on probiotic survival may be provided, for example during storage of the precursor composition.

The present invention particularly relates to microparticles that are intended to be ingested by humans, but potentially other animals. Accordingly, it will be appreciated by the skilled person that the ingredients of the microparticles of the present invention are selected such that they are fit for purpose. That is, in the case of a microparticle intended to be ingested by humans, the ingredients of the microparticle are approved for human consumption by any necessary authorities. Likewise, for products intended for animal consumption, the ingredients will be approved for such use. By way of example, the present invention is generally described with reference to microparticles intended for human consumption.

In general, components that are fit for human consumption may be considered edible or food-grade. That is, the components are intended to be consumed and they are not merely in a nontoxic form which is ancillary to their ultimate and intended purpose.

The present invention provides a product comprising the microparticles. For example, the microparticles produced in accordance with the present invention may be used in pharmaceutical or nutritional formulations (e.g. nutraceuticals), dietary supplements, functional foods and beverage products. Thus, in some embodiments, there is provided a product that has been supplemented (fortified) with the microparticles of the present invention. In some embodiments, food and beverages for humans as well as animals (e.g. pet food) may be supplemented using inventive microparticles containing one or more desirable actives, typically two actives: a hydrophobic active and a probiotic. Suitable examples of beverage products include, but are not limited to, water; milk; milk alternatives including, but not limited to, soy, rice, oat and almond “milks”; water-based beverages; milk-based beverages; carbonated beverages; non-carbonated beverages; beer; wine; and fruit and/or vegetable-based beverages.

Suitable fruit and/or vegetable-based beverages may include one or more fruit extracts and/or vegetable extracts. An extract includes juice, nectar, puree and/or pulp of or from the relevant fruit or vegetable. The extract may be fresh, raw, processed (e.g. pasteurized) or reconstituted. The one or more fruit extracts may be selected from, but are not limited to, the group comprising apple juice, pineapple juice, one or more citrus fruit juices (i.e. one or more juices of orange, mandarin, grapefruit, lemon, tangelo, cumquat, etc.), cranberry juice, noni juice, acai juice, goji juice, blueberry juice, blackberry juice, raspberry juice, pomegranate juice, grape juice, apricot juice or nectar, peach juice or nectar, pear juice, mango juice, passionfruit juice and guava puree. The one or more vegetable extracts may be selected from, but are not limited to, the group comprising aloe vera juice, beet juice, carrot juice, celery juice, kale juice, spinach juice, tomato juice and wheat grass juice. Furthermore, vegetable extracts may include extracts of herbs or spices, such as ginger juice.

Up to 10 grams of microparticles may be added per kilogram or per litre of product to be supplemented. For example, from about 7 grams to about 9 grams of microparticles may be added per kilogram or per litre of product to be supplemented. About 8 grams of microparticles may be added per kilogram or per litre of product to be supplemented. In some embodiments, 8 grams of microparticles are added per litre of juice, such as fresh orange juice, to be supplemented.

In some supplemented products, such as a liquid sweet formulation, the amount of microparticles in the product may represent up to 13% of the product weight.

In some embodiments, the product supplemented with the microparticles is a powder. For example, in some embodiments the product is a meal replacement protein powder. In these embodiments, microparticles may be added to the powder product at a microparticle:powder (i.e. other product components) ratio, by weight, of up to 1:9. For example, in some embodiments, microparticles are added at a ratio of 1:49. In some other embodiments, the ratio is about 1:9.

In some embodiments, the primary constituent of the product will be the microparticles. In some such embodiments, the microparticles may be 51% or more of the product weight. For example, products in which up to 72% of the product weight is microparticles may be produced. Such products may be pharmaceutical or nutritional formulations (e.g. nutraceuticals).

The microparticles are typically spherical. Accordingly, they often have diameters in the order of 10 μm to 50 μm. In general, the microparticles have at least one dimension that is less than 1000 μm. However, the microparticles of the present invention are typically small and have at least one dimension that is less than 50 μm.

The microparticles according to the present invention may be manufactured by providing a cross-linkable microparticle precursor composition in a finely divided state and contacting it with a cross-linking reagent. Herein the term “cross-link” (and variations thereof) generally refers to a chemical link between two or more polymeric chains of atoms. A cross-linkable microparticle precursor in accordance with the invention comprises a cross-linkable reagent. The cross-linkable reagent can be a molecule, typically a polymer incorporating repeat units, that includes groups or moieties that can be cross-linked. Cross-links bind molecules together into a network, forming a larger molecular superstructure. The cross-links may be ionic, dative, complexation and coordination linkages, covalent, and may also involve hydrogen-bonding interactions. Thus, an ionically cross-linkable polymer, therefore, is generally a polymeric molecule that is capable of forming cross-links by reaction with an ionically cross-linking reagent so as to form microparticles. Ionic cross-links may be reversible or irreversible. An ionically cross-linkable reagent has one or more ionisable groups. The term “ionisable group” refers to a chemical moiety capable of partial or full ionisation.

The cross-linkable reagent may be a basic polyelectrolyte (poly base), basic ionomer, acidic polyelectrolyte (poly acid), or an acidic ionomer. In some embodiments, the cross-linkable reagent is selected from one or more anionic monomers or polycations. As used herein, the term “polycation” or related terms such as “cationic polymer” refer to a polymer composed of positively charged macromolecules. In some embodiments, the cross-linkable reagent is selected from one or more anionic monomers or polyanions.

Suitable cross-linkable polymers may be selected from the class of hydrogels including hydrocolloids. Hydrocolloids are hydrophilic polymers, of vegetable, animal, microbial or synthetic origin, that generally contain many hydroxyl groups and may be polyelectrolytes. Hydrocolloids which are not ionically cross-linkable may be used in blends with polymers which are ionically cross-linkable.

Polymers which may be used in the present invention include but are not limited to one or a mixture of polymers selected from the group consisting of polyvinyl alcohol, alginates, carrageens, pectins, carboxy methyl cellulose, hyaluronates, heparins, heparin sulfates, heparans, chitosans, carboxymethyl chitosan, agar, gum arabic, pullulan, gellan, xanthan, tragacanth, carboxymethyl starch, carboxymethyl dextran, chondroitins including chondroitin sulfate, dermatans, cationic guar and locust bean, konjac, gum ghatti, xyloglucans, karaya gums, cationic starch as well as salts and esters thereof.

Exemplary anionic polymers include one or a mixture of alginates, pectins, carboxy methyl cellulose, hyaluronates. Exemplary cationic polymers include chitosan, cationic guar, and cationic starch.

The ionically cross-linkable polymers from which the microparticles of this invention may be produced may be functionalised with carboxylic, sulfate, phosphate, sulphonamido, phosphonamido, hydroxy and amine functional groups.

The cross-linking reagent may be as a solution of an inorganic salt. Generally, suitable cross-linking reagents are solutions of dissolved ions. The cross-linking ions used to cross-link the cross-linkable reagent may be anions or cations depending on whether the cross-linkable reagent is anionically or cationically cross-linkable. Appropriate bio-compatible cross-linking ions include but are not limited to cations selected from the group consisting of calcium, magnesium, barium, strontium, zinc, boron, beryllium, aluminium, iron, copper, cobalt, nickel, lead and silver ions, or mixtures of any two or more thereof. Anions may be selected from but are not limited to the group consisting of carboxylate, phosphate, sulphate, oxalate, bicarbonate, and carbonate ions. More broadly, the anions are derived from polybasic organic or inorganic acids. Preferred cross-linking cations are calcium ions.

In a preferred embodiment of the present invention, formation of the microparticles takes place by a sol-gel phase transition and the reagents used in this embodiment should be selected accordingly. Thus, in principle the cross-linkable reagent blended with other components of the microparticle precursor composition and cross-linking reagent may be selected from any suitable combination that will result in formation of microparticles by a sol-gel phase transition associated with the cross-linkable reagent. This said, the compatibility of the reagents with active(s) (typically, a probiotic and a hydrophobic active) incorporated into the microparticle, and the release characteristics of such actives when present in the microparticles will also need to be considered. Accordingly, the selection of the reagents will also depend upon the ultimate use of the microparticle and other components may be included in order to optimise the stability of the active(s) for the intended period of use.

The present invention will be described for the purposes of (non-limiting) illustration with reference to the use of alginates as the cross-linkable reagent. Alginates are particularly preferred for use in the invention because they are physiologically acceptable and they form thermally stable gels after binding with a suitable cation. Ionic gelation of alginates is based on their affinity towards and ability to bind certain ions. Alginates form strong, stable gels with divalent cations such as Ca²⁺, Sr²⁺, Zn²⁺, Co²⁺ and Ba²⁺. Trivalent cations, such as Fe³⁺ and Al³⁺, may also effect gelling. There is no gelation with monovalent cations.

The use of alginate gels in the present invention is also advantageous since these gels may exhibit desirable active release characteristics. For example, alginate gels show stability in low pH conditions as they shrink and do not swell and disintegrate. Active release is therefore also low. On the other hand, alginate gels swell rapidly and show dissolution and/or disintegration in weak alkaline conditions. This property enables alginate gels to be used effectively to deliver a probiotic and a hydrophobic active to the human intestine (pH above 6.7). Alginate gels are also muco-adhesive and tend to stick to the intestinal mucosa for prolonged periods. Thus, the use of alginate gels may be particularly advantageous for the delivery of certain actives, such as probiotics.

A number of factors influence alginate gel formation and these may need to be considered when implementing the present invention. One factor is the prevalence and length of gluconate (G) residues against the prevalence and length of mannuronate (M) residues. The M/G ratio is an important factor, at least in relation to Ca²⁺ cross-linking. As the M/G ratio decreases, the requirement for Ca²⁺ ion concentration increases for effective cross-linking. Gels formed from alginates with a high G content also tend to be stiffer, more brittle, and more porous and maintain strength and integrity for longer periods of time. Such gels do not swell excessively on cross-linking. Alginates with a high M content tend to form softer, less porous elastic gels with high shrinkage. Such gels swell more, dissolve more easily and increase in size more than high G content alginate gels. The gel strength also increases with increases in alginate concentration and with higher G content.

With reference to using CaCl₂ as a cross-linking reagent, Ca Cl₂ concentrations up to about 0.2M may be used, for example from about 0.1M to 0.2M, such as about 0.1M.

One benefit associated with the use of alginates is that the gel's thermal uses are stable and independent of temperature (up to the boiling point of water). However, the kinetics of gelling can be modified by adjusting the prevailing temperature as might be necessary.

These factors, and others, may be manipulated to achieve the desired outcomes with respect to gel formation and gel properties. These kinds of factors will also need to be considered when using other types of cross-linkable and cross-linking reagents.

In certain embodiments of the present invention, the cross-linkable reagent may be a blend of an alginate and a pectin. Pectin is a biodegradable acidic carbohydrate polymer which is commonly found in plant cell walls. Pectin can consist of an α-(1→4)-linked polygalacturonic acid and rhamnose residue backbone that may be modified with neutral sugar side chains and non-sugar components such as methyl and acetyl groups. The extent of rhamnose insertions along the α-(1→4)-linked polygalacturonic acid backbone and other modifications vary depending on plant sources. The galacturonic acid content is generally more than 70% whereas the rhamnose content is typically <2%. Rhamnose residues are α-(1→2)-linked to galacturonic acid residues in the backbone. They cause the formation of a T-shaped kink in the backbone chain, and increases in rhamnose content lead to more flexible molecules. The neutral sugar side chains are attached to the rhamnose residues in the backbone at the O-3 or O-4 position. The rhamnose residues tend to cluster together on the backbone.

Methylation occurs at the carboxyl groups of galacturonic acid residues. The degree of methylation or methyl-esterification is defined as the percentage of carboxyl groups (galacturonic acid residues) esterified with methanol. Based on the degree of methylation or methyl-esterification, pectins are divided into two classes, low methoxyl pectin with a degree of methylation or methyl-esterification of <50% and a high methoxyl pectin with a degree of methylation or methyl-esterification of >50%.

Both high and low methoxyl form gels. However, these gels form via different mechanisms. High methoxyl pectin forms a gel in the presence of high concentrations of co-solutes (sucrose) at low pH. Low methoxyl pectin forms a gel in the presence of divalent cations such as Ca²⁺, Sr²⁺, Zn²⁺, Co²⁺ and Ba²⁺, Ca²⁺ ions in particular. The divalent cations-low methoxyl pectin gel network is built by formation of what is commonly referred to as an “egg-box” junction zone, in which divalent cations cause the cross-linking of two stretches of polygalacturonic acid chains.

High methoxyl pectins are generally not reactive with divalent cations and therefore cannot form a divalent cation gel. However, certain high methoxyl pectins have been reported to be calcium sensitive and capable of calcium gel formation. In addition, high methoxyl pectins can be made calcium-reactive by a block wise de-esterification process while still having a degree of methylation or methyl-esterification of >50%.

Accordingly, low methoxyl pectins are generally preferred for embodiments of the present invention where a blend of alginate and pectin is used as the cross-linkable reagent. In this way, the same cross-linking reagent can be used for both the alginate and the pectin, when this combination is used. For example, the cross-linking reagent may be CaCl₂. Indeed, low methoxyl pectin may have a higher affinity for calcium, so the combination of low methoxyl pectin and alginate may lead to a cross-linked matrix with improved gel strength when compared to one of cross-linked alginate alone.

Calcium-low methoxyl pectin gel formation is influenced by several factors, including degree of methylation or methyl-esterification, ionic strength, pH, and molecular weight. The lower the degree of methylation or methyl-esterification and the higher the molecular weight, the more efficient the gelation. Furthermore, the calcium-low methoxyl pectin gelation is more efficient at a neutral pH of about 7.0 than about 3.5. Lastly, the addition of monovalent cations (e.g. the addition of NaCl to provide Na⁺) enhances the gelation, i.e., less calcium is required for gel formation.

Low methoxyl pectins are typically obtained through a chemical de-esterification process. Commercial low methoxyl pectins typically have a degree of methylation or methyl-esterification of 20-50%. Completely de-esterified pectin can be referred to as “pectic acid” or “polygalacturonic acid”. Pectic acid in the acid form is insoluble but is soluble in the salt form. The common salt form of pectic acid is either a sodium or potassium salt.

Commercial pectins are mainly derived from citrus and apples. However, apart from citrus and apples, pectins can also be isolated from many other plants, such as aloe vera. Aloe vera leaves consist of two parts, an outer green rind and a clear inner gel which is also referred to as pulp. Aloe pectin is extracted from the inner gel or outer rind cell wall fibres. Use of a chelating agent at a slight alkaline pH is found to be the most efficient extraction method. Aloe vera pectin is naturally a low methoxyl pectin, having a degree of methylation or methyl-esterification generally <30% that can be as low as <10%, and is capable of divalent cation gelation. A monovalent cation, such as Na⁺, K⁺ or Li⁺ accelerates the formation of the gel. In addition, aloe vera pectin possesses several unique chemical properties that are particularly related to gelation. For example, it has a high molecular weight of >1×10⁶ Da and a high intrinsic viscosity of >550 ml/g. Also, it has a high rhamnose content of >4%, which is at least twice the content of other pectins derived from plants such as citrus, apple, sugar beet, and sunflower. Rhamnose is a key sugar in the pectin backbone and its content affects the flexibility of the molecule. Aloe vera pectin also possesses a rare sugar, 3-OMe-rhamnose which has not been described in any other pectins. The galacturonic acid content of aloe vera pectin is >70% and can be as high as >90%. Due to its characteristics, aloe vera pectin may be a preferred pectin for some embodiments of the present invention.

Other combinations of cross-linkable reagent and cross-linking reagent may be used in the present invention including: chitosan+tripolyphosphate, carboxymethylcellulose+Al³⁺, k-carrageenan+K⁺, k-carrageen+NH₄⁺, pectin+Ca²⁺, gelan gum+Ca²⁺, and polyphosphazene+Ca²⁺.

The cross-linkable reagent generally comprises a cross-linkable polymer, such as an alginate or a pectin, in a solution with solvent, such as water or an aqueous solution. Typically, the concentration of cross-linkable polymer in the solution will be from about 5% w/w to about 15% w/w, preferably from about 8% w/w to about 12% w/w, preferably about 10% w/w. The quantity of cross-linkable reagent used may be such that the concentration of cross-linkable polymer in the microparticle precursor composition may be from about 2% w/w to about 8% w/w, preferably from about 3% w/w to about 6% w/w, preferably from about 3% w/w to about 4% w/w. For example, in embodiments where the cross-linkable reagent is a blend of an alginate and a pectin, the concentration of sodium alginate in the microparticle precursor composition may be about 2% w/w and the concentration of pectin may also be about 2% w/w. In some other embodiments where the cross-linkable reagent is a blend of an alginate and a pectin, the concentration of sodium alginate in the microparticle precursor composition may be about 2% w/w and the concentration of pectin may be about 1% w/w. In some embodiments, the concentration of sodium alginate in the microparticle precursor composition may be about 2% w/w and the concentration of pectin may be between about 1% w/w and 2% w/w.

As the cross-linkable reagent is generally provided in or as an aqueous solution, hydrophobic actives will typically be immiscible in the cross-linkable reagent and it can be challenging to incorporate a hydrophobic active and a probiotic into a microparticle. To address this problem, the present invention provides a microparticle precursor composition comprising a blend of a probiotic, a cross-linkable reagent and an emulsion comprising a hydrophobic active. This precursor may be used to provide a microparticle comprising a hydrophobic active and a probiotic distributed within a cross-linked matrix. Central to this aspect of the invention is combining the hydrophobic active and the probiotic into the microparticle precursor composition in such a way that the viability of the probiotic is substantially maintained so that a microparticle with viable probiotic can be manufactured.

It can be undesirable to combine the hydrophobic active, probiotic and cross-linkable reagent by emulsifying these three components together as the shear forces generated in emulsification processes may result in probiotic cell disruption and losses in probiotic viability. The use of the emulsion comprising the hydrophobic active in accordance with the present invention can avoid this problem.

Probiotics are defined as live microbes that beneficially affect the human or animal that has ingested it by modulating mucosal and systemic immunity, as well as improving intestinal function and microbial balance in the intestinal tract. Probiotics can exhibit one or more of the following non-limiting characteristics: non-pathogenic or non-toxic to the host; are present as viable cells, preferably in large numbers; capable of survival, metabolism, and persistence in the gut environment (e.g., resistance to low pH and gastrointestinal acids and secretions); adherence to epithelial cells, particularly the epithelial cells of the gastrointestinal tract; microbicidal or microbistatic activity or effect toward pathogenic bacteria; anticarcinogenic activity; immune modulation activity, particularly immune enhancement; modulatory activity toward the endogenous flora; enhanced urogenital tract health; antiseptic activity in or around wounds and enhanced would healing; reduction in diarrhoea; reduction in allergic reactions; reduction in neonatal necrotizing enterocolitis; reduction in inflammatory bowel disease; and reduction in intestinal permeability.

The probiotic used as an active in the present invention may be selected from, but not limited to, the group consisting of yeasts such as Saccharomyces, Debar Candida, Pichia and Torulopsis, moulds such as Aspergillus, Rhizopus, Mucor, and Penicillium and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus, as well as combinations thereof. Examples of suitable probiotics include: Saccharomyces cereviseae (boulardii), Bacillus coagulans, Bifidobacterium animalis, Bifidobacterium bifidum, Bifidobacterium breve, Bifidobacterium infantis, Bifidobacterium longuin, Bifidobacterium lactis, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus fermentum, Lactobacillus gasseri, Lactobacillus helveticus, Lactobacillus johnsonii, Lactobacillus paracasei, Lactobacillus plantarum, Lactobacillus reuteri, Lactobacillus rhamnosus (Lactobacillus GG), Lactobacillus sake, Lactobacillus salivarius, Lactococcus lactis, Pediococcus acidilactici, Pediococcus pentosaceus, Pediococcus acidilactici, Pediococcus halophilus, Streptococcus faecalis, Streptococcus thermophilus and Saccharomyces boulardii. More specifically the probiotic may selected from the group comprising of Lactobacillus casei Lc431, Lactobacillus rhamnosus CGMCC 1.3724, Bifidobacterium lactis BB12, Bifidobacterium lactis CNCM I-3446, Bifidobacterium longum ATCC BAA-999, Lactobacillus paracasei CNCM I-2116, Lactobacillus johnsonii CNCM I-1225, Lactobacillus fermentum VRI 003, Bifidobacterium longum CNCM I-2170, Bifidobacterium longum CNCM I-2618, Bifidobacterium breve, Lactobacillus paracasei CNCM I-1292, Lactobacillus rhamnosus ATCC 53103, Enterococcus faecium SF 68, Lactobacillus reuteri ATCC 55730, Lactobacillus reuteri ATCC PTA 6475, Lactobacillus reuteri ATCC PTA 4659, Lactobacillus reuteri ATCC PTA 5289, Lactobacillus reuteri DSM 17938, and mixtures thereof. In some preferred embodiments, the microparticle may contain Lactobacillus casei Lc431 or Bifidobacterium lactis BB12.

The probiotic is viable if it is alive and capable of reproduction or colonization. The concentration of viable probiotics in the microparticle must exceed a certain threshold value or the beneficial effect of the probiotics is not provided. Quantities of probiotics are typically evaluated in terms of colony forming units (CFU). Typically, dosages of about one to two million CFU are required for adult humans to receive the beneficial effects of the probiotic. So that these sorts of dosages may be achieved, the loading of viable probiotic in the microparticle is often in the order of five to ten billion CFU/g, for example about 2.5% to about 5% of the microparticle weight. In some embodiments, the loading of probiotic is around 2.5% of the microparticle weight. In some other embodiments, the loading of probiotic may be around 4% of the microparticle weight.

Use of the present invention can improve probiotic survival during microparticle production. Improvement in probiotic survival is expressed as a percentage and calculated according to Formula 1 below.

$$Improvement in Probiotic Survival ( \% ) = 100 - ( 100 \times \frac{{log}_{10} ( \begin{matrix}number of CFU per unit weight or \\ unit volume of the comparative product\end{matrix} )}{{log}_{10} ( \begin{matrix}number of CFU per unit weight or \\ unit volume of the product of \\ the present invention\end{matrix} )} ) \qquad Formula 1$$

In some embodiments, the improvement in probiotic survival may be around 20% to around 50%, when compared to processes in which the hydrophobic active, probiotic and cross-linkable reagent are emulsified together. For example, probiotic survival may be improved by around 30%.

Hydrophobic actives are active compounds that are generally immiscible in water. These actives may be lipids or actives that are provided in a solution with a water immiscible solvent. This water immiscible solvent may be a lipid.

Hydrophobic actives that are lipids include nutritional oils, such as fish oil. As used herein, the term “fish oil” means oil derived from fish and/or other marine organism(s). For example, fish oil includes oil derived from krill, calamari (squid), caviar, abalone scallops, anchovies, catfish, clams, cod, herring, lake trout, mackerel, menhaden, orange roughy, salmon, sardines, pilchards, sea mullet, sea perch, shark, shrimp, trout and tuna, and combinations thereof.

Fish oil is a source of omega-3 fatty acid. Other sources of omega-3 fatty acid include, but are not limited to, plant-based oils that are rich in omega-3 fatty acids such as, walnut, linseed (flaxseed), rapeseed (canola), chia (typically Salvia hispanica) seed and hemp seed oils. Thus, sources of omega-3 fatty acids may be hydrophobic actives for the purposes of the present invention.

When a source of omega-3 fatty acid is a fish oil or plant-based oil, the oil may be a crude oil, a partially refined oil, a refined oil, or an oil concentrate.

In some embodiments, such as when the active is a source of omega-3 fatty acid (e.g. fish oil), the amount of hydrophobic active in the microparticle may represent up to 20% of the microparticle weight. In some embodiments, the amount of hydrophobic active may be around 10% of the microparticle weight.

The term “omega-3 fatty acid” means a long chain polyunsaturated fatty acid having a carbon-carbon double bond between the third and fourth carbon from the methyl terminus of the fatty acid chain. Common omega-3 fatty acids include alpha linolenic acid (C18:3; (9Z,12Z,15Z)-Octadeca-9,12,15-trienoic acid, “ALA”), eicosapentaenoic acid (C20:5; (5Z,8Z,11Z,14Z,17Z)-icosa-5,8,11,14,17-pentaenoic acid, “EPA”), and docosahexaenoic acid (C22:6; (4Z,7Z,10Z,13Z,16Z,19Z)-docosa-4,7,10,13,16,19-hexaenoic acid, “DHA”). Other common omega-3 fatty acids include, but are not limited to, stearidonic acid (C18:4), eicosatetraenoic acid (C20:4), and docosapentaenoic acid (C22:5).

Other oils that are hydrophobic actives for the purposes of the present invention include, but are not limited to, avocado oil, apricot kernel oil, argan oil, evening primrose oil, garlic oil and peppermint oil.

The hydrophobic active may be a lipid-soluble vitamin. Lipid-soluble vitamins which may be used in this invention include vitamins A, vitamins D, vitamins E, vitamins K, and ubiquinones, for example.

The vitamins A include vitamins A such as retinol (vitamin A₁ alcohol), retinal (vitamin A₁ aldehyde), vitamin A₁ acid, 3-dehydroretinol (vitamin A₂ alcohol), and 3-dehydroretinal (vitamin A₂ aldehyde) and provitamins A such as β-carotene (β,β-carotene), α-carotene (β,ε-carotene) and γ-carotene (β,ψ-carotene), for example. A provitamin A, such as β-carotene, may be a particularly preferred active for incorporation into the microparticle of the present invention. In some embodiments, β-carotene may be used in combination with a probiotic and fish oil.

The vitamins D include vitamins D such as vitamin D₂, vitamin D₃, vitamin D₄, vitamin D₅, vitamin D₆, and vitamin D₇ and provitamins thereof, for example.

The vitamins E include tocopherols such as α-tocopherol, β-tocopherol, γ-tocopherol, and δ-tocopherol and tocotrienols such as α-tocotrienol, β-tocotrienol, γ-tocotrienol, and δ-tocotrienol, for example.

The vitamins K include vitamin K₁ and vitamins K₂, for example.

The ubiquinones include ubiquinone-1 to ubiquinole-12 (Q-1 to Q-12) and the oxidized forms thereof and amino chloride compounds thereof, for example.

Hydrophobic actives, such as lipid-soluble vitamins, will typically be dissolved in a lipid (acting as a water immiscible solvent) in order to put them into a form suitable for use in the present invention. Lipids may be oils, waxes, fatty acids, fatty alcohols, monoglycerides and triglycerides, which are either saturated or unsaturated. In some embodiments, a blend of lipids may be used.

In general, the lipid or lipids selected for dissolving the hydrophobic active will be liquid. That is, a lipid that has a melting point of 25° C. or less, preferably 10° C. or less. In some embodiments, it is preferred that the lipid has a melting point lower than the storage temperature of the microparticle. Liquid lipids are often selected as' they may be more readily emulsified with other components to form the emulsion comprising the hydrophobic active. Solid lipids may need to be heated to above their melting temperature or dissolved in a suitable solvent, which may be another lipid, in order to be effectively combined with the active. Typically, if solid lipids are used, they are first blended with a suitable solvent (such as a liquid lipid) so as to produce a lipid mixture that is liquid at 25° C. or less, preferably 10° C. or less.

Liquid lipids may also be more readily digested by the human or animal ingesting the microparticle. Thus, the selection of a liquid lipid may be useful to ensure that any actives dissolved in the lipid are released at an optimum time.

Lipids used in embodiments of the invention can be derived from many different sources. In some embodiments, lipids used in embodiments of the invention can include biological lipids. Biological lipids can include lipids (fats or oils) produced by any type of plant, such as vegetable oils, or animal. In one embodiment, the biological lipid used includes triglycerides.

Many different biological lipids that are derived from plants may be used, and these plants may be genetically modified crops. By way of example, plant-based lipids can include soybean oil, canola oil, cottonseed oil, grape seed oil, mustard seed oil, corn oil, linseed oil, safflower oil, sunflower oil, poppy seed oil, pecan oil, walnut oil, peanut oil, rice bran oil, camellia oil, olive oil, palm oil, palm kernel oil and coconut oil, or combinations thereof. Other plant-based lipids can be obtained from almond, argan, avocado, babassu, beech, ben (from the seeds of the Moringa olejfera), Borneo tallow nut, brazil nut, camelina, caryocar (pequi), cashew nut, cocoa, cohune palm, coriander, cucurbitaceae (e.g. butternut squash seed oil, pumpkin seed oil and watermelon seed oil), hemp, kenaf, macadamia, noog abyssinia, perilla, pili nut, quinoa, sacha inchi, seje, sesame, shea nut, tea seed and papaya seed. These may be used alone or in combination with another lipid.

Lipids derived from animals may also be used, for example, white grease, lard (pork fat), tallow (beef fat), anhydrous milk fat, and/or poultry fat may be used. However, as noted above, liquid lipids with a melting point of 25° C. or less are preferred.

The lipid may be synthetic triglyceride of the formula

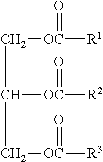

wherein R¹, R² and R³ may be the same or different and are aliphatic hydrocarbyl groups that contain from 7 to about 23 carbon atoms. The term “hydrocarbyl group” as used herein denotes a radical having a carbon atom directly attached to the remainder of the molecule. The aliphatic hydrocarbyl groups include the following:
(1) Aliphatic hydrocarbon groups; that is, alkyl groups such as heptyl, nonyl, undecyl, tridecyl, heptadecyl; alkenyl groups containing a single unsaturated bond such as heptenyl, nonenyl, undecenyl, tridecenyl, heptadecenyl, heneicosenyl; alkenyl groups containing plural unsaturated bonds; and all isomers thereof.
(2) Substituted aliphatic hydrocarbon groups containing non-hydrocarbon substituents, such as hydroxy of carbalkoxy groups.
(3) Hetero groups; that is, groups which, while having predominantly aliphatic hydrocarbon character, contain atoms other than carbon, such as oxygen, nitrogen or sulfur, present in a chain or ring otherwise composed of aliphatic carbon atoms.

Many biological lipids need to be processed following extraction from their natural source in order to remove impurities. For example, the lipids may be degummed to remove phospholipids, bleached to remove impurities and minor components such as chlorophyll and carotenoids that can give colour to the oil and fractionated to remove the free fatty acids that can give an undesirable taste and/or smell to the refined oil. “Fractionating” and related terms, as used herein, refer to a process in which less volatile components are separated from more volatile components, typically comprising the separation of triglycerides from free fatty acids in plant-derived biological lipids (oils).

Processing can include hydrogenation of the lipid. In this process, the lipid is hydrogenated by reducing the unsaturated bonds in the lipid. This usually achieved by exposing the lipid to hydrogen in the presence of a catalyst, such as a nickel catalyst. Hydrogenation may be complete or partial. A partially hydrogenated lipid may include a blend of unhydrogenated lipid and fully hydrogenated lipid.

Hydrogenating the lipid can be advantageous as it reduces the lipid's sensitivity to oxidation. Some lipids are particularly susceptible to oxidation, leading to them going rancid and producing an objectionable flavour, and hydrogenation of these lipids may be useful. However, hydrogenation can increase the melting point of the lipid, thus transforming a liquid lipid into a solid one, which can affect the ease with which the lipid may be blended with other components of the composition. Accordingly, the degree to which a lipid may be hydrogenated will be selected bearing in mind the impact any increase in melting point will have on the ease with which the lipid can then be used to dissolve the hydrophobic active:

Preferably, the lipid may be a plant-based lipid selected from the group consisting of almond oil, canola oil, cod liver oil, corn oil, cotton seed oil, flaxseed oil, grape seed oil, peanut oil, safflower oil, sesame oil, soybean oil, sunflower oil, walnut oil, coconut oil or palm kernel oil. It will appreciated that, in some embodiments, a hydrophobic active (for example a lipid-soluble vitamin) may be dissolved in a lipid that constitutes a hydrophobic active in of itself (for example an oil rich in omega-3 fatty acids, such as flaxseed oil).

The emulsion comprising the hydrophobic active may be formed by combining the hydrophobic active with a suitable liquid that is readily miscible with the cross-linkable reagent, but not readily miscible with the hydrophobic active. Typically, the hydrophobic active is emulsified with water using a suitable emulsifier, with this emulsion being readily miscible with the cross-linkable reagent being used. However, in some embodiments, the liquid may be the cross-linkable reagent.

The hydrophobic active and the liquid may be emulsified together using conventional emulsification techniques that will be known to those skilled in the art. In some embodiments, the emulsion may require further refining to achieve an emulsion in which the droplets of hydrophobic active are of a suitably small size. For example, multiple emulsification or homogenization steps may be required to refine the droplets of hydrophobic active to a suitably small size. A suitably small droplet size may facilitate a more even distribution of the hydrophobic active through the cross-linked matrix of the microparticle. In some embodiments, the distribution of hydrophobic active droplets through the microparticle may be substantially uniform. Furthermore, with reductions in droplet size, the surface area to volume ratio of the hydrophobic active increases. Accordingly, there is more surface area available for the hydrophobic active to contact other components of the microparticle. The droplet size in the emulsion will also impact on the volume of interstitial space available between droplets. As noted above it is believed that the probiotic may advantageously be located within the interstitial spaces. A suitably small droplet size may be from 0.5 to 1 μm.

Certain hydrophobic actives, such as fish oil, are susceptible to oxidation. To prevent and/or delay onset of oxidation, the emulsion comprising the hydrophobic active may be formed in an inert atmosphere, such as a nitrogen or argon atmosphere, to reduce or prevent exposure to oxygen.

An emulsifier is used in order to enhance the stability of the emulsion comprising the hydrophobic active. The emulsifier may be any food-grade surface active ingredient, cationic surfactant, anionic surfactant and/or amphiphilic surfactant. Such emulsifiers can include one or more of, but are not limited to, lecithin, modified lecithin, chitosan, modified starches (e.g., octenylsuccinate anhydride starch), pectin, gums (e.g., locust bean gum, gum arabic, guar gum, etc.), alginic acids, alginates and derivatives thereof, cellulose and derivatives thereof, distilled monoglycerides, mono- and diglycerides, diacetyl tartaric acid esters of mono- and diglycerides (DATEM), polysorbate 60 or 80 (TWEEN 60 or 80), sodium stearyl lactylate, propylene glycol monostearate, succinylated mono- and diglycerides, acetylated mono- and diglycerides, propylene glycol mono- and diesters of fatty acids, polyglycerol esters of fatty acids, lactylic esters of fatty acids, glyceryl monosterate, propylene glycol monopalmitate, glycerol lactopalmitate and glycerol lactostearate, and mixtures thereof. In some embodiments, lecithin is used as an emulsifier.

The emulsifier may be added at a ratio of emulsifier:liquid of about 1:50 to about 1:15, preferably of about 1:45 to about 1:25. In some embodiments, the ratio used is about 1:29 on a weight basis. In some other embodiments, the ratio used is about 1:39.

Typically, the emulsifier is blended with the at least a portion of the liquid before that emulsifier mixture is emulsified with the hydrophobic active. However, in some embodiments, the emulsifier, liquid and hydrophobic active may be mixed together simultaneously. In some embodiments, the emulsifier is mixed with the hydrophobic active prior to that mixture being emulsified with the liquid.

In some embodiments, the emulsifier is blended with around 20% to 50% (by weight), for example around one quarter or one third, of the liquid before that emulsifier mixture is mixed with the hydrophobic active and remaining liquid. The emulsifier may be added to the first portion of the liquid at a ratio of emulsifier:liquid portion of about 1:11 to about 1:7, preferably of about 1:10 to about 1:8, more preferably about 1:9 on a weight basis.

In view of the fact that the present invention relates to microparticles that are intended to be ingested by humans, in some embodiments, the mixture of emulsifier and liquid may be sterilised prior to emulsification with the hydrophobic active. The mixture of emulsifier and liquid may be sterilized by heating it to above 80° C. for a suitable length of time. For example, the mixture may be sterilized at 85° C. for 30 minutes. Often the sterilised mixture is cooled before emulsifying it with the hydrophobic active.

The hydrophobic active and mixture of emulsifier and liquid may be emulsified together at a ratio of hydrophobic active:mixture of emulsifier and liquid of from about 1:5 to about 5:1, preferably from about 10:35 to about 1:1, on a weight basis. In some embodiments, the ratio used is about 2:3 on a weight basis. In some other embodiments, the ratio used is about 1:3. In some embodiments, the ratio used is about 1:4.

Once the emulsion comprising the hydrophobic active is formed, the probiotic can be blended with the emulsion comprising the hydrophobic active to form a probiotic-containing emulsion. The probiotic may be shear-sensitive in that subjecting the probiotic to high shear forces may result in cell disruption and losses in viability. In view of this, the probiotic should be blended with and dispersed though the emulsion in such a way that the viability of the probiotic is not unduly compromised. Accordingly, blending the probiotic with the emulsion comprising the hydrophobic active, or indeed any other component of the microparticle, involves subjecting the probiotic to suitably low shear blending. Suitably low shear blending is blending that is conducted below the shear rate at which significant cell disruption and losses in probiotic viability occur. For example, low shear rates may be the types of shear rates generated by a blending impeller operating at up to 300 rpm, such as from 100 to 300 rpm, but preferably 100 rpm or less. Low shear blending includes low shear mixing. Thus, in some embodiments, blending the probiotic with the emulsion comprising the hydrophobic active may comprise mixing the probiotic with the emulsion comprising the hydrophobic active.

The probiotic-containing emulsion can then be blended with the cross-linkable reagent to form the microparticle precursor composition. It will be appreciated that the blending with the cross-linkable reagent is also suitably low shear blending to ensure that the viability of the probiotic is not unduly compromised during formation of the microparticle precursor composition. Indeed, in the present insemination, any probiotic-containing component that is used to produce the microparticle precursor composition, including any intermediate probiotic-containing mixtures, should be subjected to suitably low shear rates to ensure that the viability of the probiotic is not unduly compromised.

The hydrophobic active is typically the discontinuous phase in the emulsion comprising the hydrophobic active. By providing the hydrophobic active in the form of an emulsion with a liquid that is readily miscible with the cross linkable reagent, the liquid carries the hydrophobic active and allows it to be effectively dispersed within the cross-linkable reagent. Thus, the discontinuous droplets of hydrophobic active are typically carried through into microparticle precursor composition. Furthermore, when the hydrophobic active is dispersed within the microparticle precursor composition, the microparticle produced using the precursor can have the hydrophobic active distributed within the cross-linked matrix.

Generally, for efficiency of mixing, the probiotic is blended with the emulsion comprising the hydrophobic active before the blend comprising the probiotic and the hydrophobic active, i.e. the probiotic-containing emulsion, is blended with the cross-linkable reagent. In some embodiments, the probiotic cells may contact the emulsion droplets and become incorporated into them. This may also enhance probiotic survival. Blending the hydrophobic active with the cross-linking reagent before blending in the probiotic hampers the probiotic from effectively contacting the hydrophobic active.

The probiotic will generally be located in the continuous phase of the probiotic-containing emulsion. Likewise, the probiotic will typically be located in the continuous phase of the microparticle precursor composition. Most of the probiotic cells may be located in the interstitial spaces between the discontinuous hydrophobic active phase. The discontinuous droplets of hydrophobic active may be densely packed so that the interstitial spaces are shielded from the external environment. Without being bound by theory, it is believe that the probiotic is protected within the interstitial spaces so that its survival in the probiotic-containing emulsion and microparticle precursor composition is improved. Thus, it has been found that incorporating a hydrophobic active into a microparticle precursor composition in accordance with the present invention has a beneficial effect on probiotic survival during storage of the precursor composition, when compared to a microparticle precursor composition containing no hydrophobic active. For example, the microparticle precursor composition of the present invention may be stored ready for use at around 4° C. for two to three months with the probiotic survival being maintained at 90 to 98%. Probiotic survival is calculated according to Formula 2 below.

$$Probiotic Survival ( \% ) = 100 \times \frac{{log}_{10} ( \begin{matrix}final number of CFU per unit \\ weight or unit volume\end{matrix} )}{{log}_{10} ( \begin{matrix}initial number of CFU per unit \\ weight or unit volume\end{matrix} )} \qquad Formula 2$$

The beneficial improvements in probiotic survival in the microparticle precursor composition may still be attained if the emulsion is formed using a lipid rather than a hydrophobic active. Accordingly, in another embodiment, the present invention provides a microparticle precursor composition comprising a blend of a probiotic, a cross-linkable reagent and an emulsion comprising a lipid. Suitable lipids may include those described above as being suitable for dissolving hydrophobic actives in order to put them into a form suitable for use in the present invention.

The invention further provides a method of producing a microparticle precursor composition comprising: blending an emulsion comprising a lipid with a probiotic to form a probiotic-containing emulsion; and blending the probiotic-containing emulsion with a cross-linkable reagent. Both blending steps will be performed at suitably low shear rates to ensure that probiotic viability is not unduly compromised.

The present invention also provides a microparticle comprising a lipid and a probiotic distributed within a cross-linked matrix.

The dispersed arrangement of the hydrophobic active (or lipid) and probiotic in the microparticle precursor composition may be carried through to the final microparticle. Thus, the hydrophobic active or lipid can be distributed within the cross-linked matrix of the microparticle as a discontinuous phase. In some embodiments the droplets of hydrophobic active or lipid are distributed through the matrix with a substantially uniform distribution. In some other embodiments, the distribution may lead to portions within cross-linked matrix having a higher proportion of hydrophobic active or lipid than other portions.

Furthermore, the probiotic may be distributed within the cross-linked matrix of the microparticle so that it is located within the interstitial spaces between the droplets of hydrophobic active or lipid. Accordingly, the probiotic may be protected within the interstitial spaces between the hydrophobic active or lipid droplets in the microparticle. This may lead to an improvement in probiotic survival of 20% to 50%, for example from 30% to 50%. The distribution of the probiotic within the cross-linked matrix may be a substantially uniform distribution throughout the matrix.

In summary, the present invention utilises an emulsion comprising a hydrophobic active in order to permit the hydrophobic active to be blended with the probiotic and the cross-linkable reagent so as to form the microparticle precursor composition without subjecting the probiotic to stresses, namely shear forces, that will result in cell disruption and losses in viability.

Moreover, the present invention may provide a shielding structure formed of droplets of hydrophobic active or of lipid that improves survival of the probiotic within the interstitial spaces between the droplets both in the microparticle precursor composition and the ultimate microparticle.

The present invention also provides a method of producing a microparticle comprising providing a microparticle precursor composition in accordance with the present invention in a finely divided state; and exposing the finely divided microparticle precursor composition to a cross-linking reagent to thereby form microparticles.

In some embodiments, the probiotic and/or the hydrophobic active may have a flavour that is considered objectionable. In some embodiments, the probiotic and/or the hydrophobic active in the microparticle may not have a flavour that is, of itself, considered objectionable. However, it may nevertheless be desirable to mask the flavour of the probiotic and/or the hydrophobic active as it may detract from the quality of a product that the microparticle may be incorporated into. For example, the microparticle may be incorporated into dietary supplements, functional foods and beverage products and in these goods it may be desirable for the flavour of the probiotic and/or the hydrophobic active not to taint the flavour of the good. As an example, if microparticles: incorporated into orange juice to provide a supplemented (fortified) juice, it may be desirable to mask the flavour of the probiotic and/or the hydrophobic active so that the consumer does not perceive any change in the flavour of the juice as a result of the supplementation (fortification).

“Flavour” as used herein includes tastes or smells that may be perceived by the human or animal ingesting the microparticle. These flavours may be perceived by a consumer as being an objectionable flavour. An “objectionable flavour” as used herein includes tastes or smells that may be perceived by the consumer of the microparticle as being unpleasant or “off”. These flavours may be astringent, bitter, musty, chalky, reminiscent or cardboard, fishy, sulfurous (i.e. a smell or taste associated with decomposing protein), metallic, rusty and/or generally foreign. Flavours may be inherent to one or more components of the microparticle, including any actives. Alternatively or additionally, flavours may result from one or more components of microparticle partially or fully degrading.

The cross-linked matrix of the microparticle of the present invention may surround, enclose and/or immobilise droplets of the hydrophobic actives and the probiotic in such a way that the encapsulated hydrophobic active and probiotic may be protected from degradation by limiting their exposure to the external environment (e.g. heat, moisture, acid, air, light) and they may be released at a controlled rate under specific conditions as desired. In certain embodiments, the cross-linked matrix of the microparticle may mask any flavour of the active, particularly when the flavour may be perceived by the consumer as being objectionable.

That is, the cross-linked matrix of the microparticle may have suitable barrier properties for limiting exposure of any active, including the probiotic and hydrophobic active, to degrading elements from the external environment. Furthermore, certain actives may be rendered stable through encapsulation by providing a microparticle with suitable barrier properties. Also, the cross-linked matrix may have suitable barrier properties for masking any flavour of an active. The cross-linked matrix may have suitable barrier properties for preventing or suppressing leakage of the probiotic and hydrophobic active from the microparticle. By preventing or suppressing leakage, the flavour associated with the probiotic or the hydrophobic active may not be perceived by the person or animal ingesting it.

One or more components of the microparticle precursor composition may be susceptible to oxidization. Often oxidation susceptible components are actives, such as fish oil. To prevent or to reduce oxidation degradation of a susceptible component, which can lead to a loss of the beneficial activity associated with that component, certain steps of the method of producing the microparticle precursor composition may be conducted under an inert atmosphere, such as a nitrogen or argon atmosphere, to reduce or to prevent exposure to oxygen. In some embodiments, each and every step where the oxidation susceptible component or a mixture or blend containing the oxidation susceptible component is handled may be conducted under an inert atmosphere. Furthermore, in embodiments where the microparticle precursor composition contains an oxidation susceptible component, the microparticle may be produced under an inert atmosphere.

One or more components of the microparticle precursor composition may be susceptible to photodegradation. Similarly, to oxidation susceptible components, photodegradation susceptible components are often actives, such as fish oil. To prevent or to reduce photodegradation certain steps of the method of producing the microparticle precursor composition may be conducted in a darkened environment, such as a covered, opaque container, to reduce or to prevent exposure to light. In some embodiments, each and every step where the photodegradation susceptible component or a mixture or blend containing the photodegradation susceptible component is handled may be conducted in a darkened environment. In addition, in embodiments where the microparticle precursor composition contains a photodegradation susceptible component, the microparticle may be produced in a darkened environment.

One or more components of the microparticle precursor composition may be thermally sensitive and appropriate precautions can be taken during production of the microparticle precursor composition and the microparticle itself to avoid exposure to temperatures that will thermally degrade the relevant component.

The present invention provides a method of producing microparticles comprising providing the microparticle precursor composition in a finely divided state; and exposing the finely divided microparticle precursor composition to a cross-linking reagent to form microparticles. That is, the cross-linkable reagent of the microparticle precursor composition reacts with the cross-linking reagent so as to form a cross-linked matrix.

There are a number of techniques, which will be known to those skilled in the art, that may be used to provide the microparticle precursor composition in a suitably finely divided state and expose it to the cross-linking agent. The microparticle precursor compositions are particularly suited being used to produce microparticles according to the method described in International Application No. PCT/AU2008/001695 (Publication No. WO 2009/062254), the entire contents of which are incorporated herein by reference. This method is the preferred method of producing microparticles in accordance with the present invention.

Other suitable methods of providing the microparticle precursor composition in a suitably finely divided state include air atomization in which the microparticle precursor composition is extruded through a syringe pump into an air atomizer device and sprayed into a cross-linking reagent bath. Electrostatic atomization (electrospray or electrohydrodynamic atomization (EHDA)), may also be suitable. In this technique, the microparticle precursor composition is supplied to a nozzle electrode and electrified to produce droplets. The droplets are dropped down into the cross-linking reagent. Spinning disk atomization, the Vortex-Bowl Disk Atomizer System, and micronozzle arrays may also be suitable for providing the microparticle precursor composition in a suitably divided state.

Once formed, the microparticles may be separated using known techniques and devices, including centrifugal separators, clarifiers, membrane filtration and filter presses depending upon the active, microparticle size and specific gravity. If the active(s) are not heat sensitive, heat may be applied to cause syneresis of the gel in order to facilitate removal of carrier/solvent/liquid (typically water).

The microparticles may be in a form ready for use or may be added to another product as necessary. In some circumstances, trace reagents may need to be washed from the microparticles before they are used. For example, when CaCl₂ is used as the cross-linking reagent to effect gelling of an alginate (cross-linkable reagent), the microparticles may need to be washed to remove unused CaCl₂.

The microparticles may be spray dried or freeze dried with or without the presence of other carrier solids (such as maltodextrins, sugars) as necessary to provide robustness.

Microparticles formed using hydrogels may be porous, so it may be advantageous to apply a coating to the microparticle to improve its barrier properties. The microparticles of the present invention are particularly suited being coated using the coating composition described in Australian Provisional Patent Application No. 2012905167 and the coating composition described in an International patent application entitled “Coating composition” which claims priority from the aforementioned provisional application, and the contents these applications are hereby incorporated herein by reference. This coating composition is the preferred coating composition for microparticles in accordance with the present invention.

The microparticle may be coated using a variety of techniques. Suitable coating techniques include, but are not limited to, immersion coating, partial immersion coating, dipping, brushing, spin coating, flow coating and spray coating. For example, wet hydrogel microparticles may be partially immersed in a coating composition, mixed to ensure an even coating and then packaged.

The amount of coating composition used to coat a product may be equivalent to up to 50% of the weight of the microparticle to be coated. In some embodiments, the amount of coating used may be equivalent to 20 to 40% of the weight of the microparticle to be coated, preferably about 30% of the weight of the microparticle.

The microparticle may be stored at 4° C., preferably at −20° C. In some embodiments, it may be preferred to store microparticles by vacuum packing them in foil.

The following non-limiting examples illustrate embodiments of the present invention.

### Example 1

### Whey Protein Isolates (WPI) Based Matrix Additive Composition

### Preparing the WPI Mixture

### Materials:

Whey protein isolates powder—10 g
Water—90 g

### Method:

A 10% WPI solution was prepared by mixing together the WPI powder and water. The mixture was allowed to stand for 30 minutes after mixing so that the WPI could rehydrate. After standing, the 10% WPI solution was heat treated at 90° C. for 30 minutes. The resulting 10% WPI mixture was cooled before use.

### Preparing the Matrix Additive Composition

### Materials:

10% WPI Mixture as described above—60 g
Glycerol—40 g
Trehalose powder—30 g

### Method:

The materials were blended together for 5 minutes at high speed using an IKA® T25 Digital ULTRA TURRAX® high-performance single-stage dispersing machine supplied by IKA-Works, Inc. The resulting composition was then sterilised at 85° C. for 30 minutes. The matrix additive composition was cooled to room temperature before use.

### Example 2

### Fish Oil Emulsion

### Preparing the Surfactant Mixture

### Materials:

Lecithin—10 g
De-ionised Water—90 g

### Method:

The surfactant mixture was prepared by dissolving lecithin in de-ionised water at ratio 1:9, using a mixer at a medium speed, until all lecithin was dissolved. The surfactant mixture was then sterilised at 90° C. for 30 minutes.

### Preparing the Fish Oil Emulsion

### Materials:

Surfactant Mixture as described above—100 g
Omega-3 Fish Oil—200 g
Water (Sterile)—200 g

### Method:

1. The primary fish oil emulsion was prepared by weighing the components into a sterile container. The mixture was then homogenised with a Silverson Heavy Duty Laboratory mixer/emulsifier at medium speed for 5 minutes. The mixer was washed in absolute alcohol and sterile water before use. No oil droplets were visible on the surface of the emulsion.
2. The primary fish oil emulsion was passed through a two-stage Twin Panda 400 (GEA Niro Soavi) homogeniser (First Stage: 250 bars, Second Stage: 50 bars) twice to further reduce the emulsion droplet size to produce the final fish oil emulsion. The homogenisation equipment was cleaned with disinfectant and sterile water before each use.
   Note: To prevent and delay onset of fish oil oxidation, great care was taken when handling the fish oil and finished emulsion containing the omega-3 fish oil. Nitrogen gas was used to create a gas blanket to reduce oxygen exposure during preparation of the fish oil emulsion. The fish oil emulsion was also mixed in a container covered with foil to decrease light exposure.

### Example 3

### Cross-Linkable Reagent

### Preparing the Cross-Linkable Reagent

### Materials:

Sodium Alginate—20 g
Pectin—20 g
De-ionised Water—360 g

### Method:

The sodium alginate, pectin and de-ionised water were mixed together thoroughly. The cross-linkable reagent was then sterilised at 90° C. for 30 minutes the day before it was to be used.

### Example 4

### Microparticle Precursor Composition—Lactobacillus casei Lc431

Preparing the Microparticle Precursor Composition—Lactobacillus casei Lc431

### Materials:

Matrix Additive Composition of Example 1—130 g
Frozen Concentrate of Lactobacillus casei Lc431—25 g
Fish Oil Emulsion of Example 2—500 mL
Cross-linkable Reagent of Example 3—400 g

### Method:

The frozen concentrate of Lactobacillus casei Lc431 was melted in a sterile container at room temperature. Then, the microparticle precursor composition was prepared by combining the sterile, prepared compositions listed below in a sterile container in following order:
1. Matrix Additive Composition of Example 1
2. Melted Concentrate of Lactobacillus casei Lc431
3. Fish Oil Emulsion of Example 2
4. Cross-linkable Reagent of Example 3

The microparticle precursor composition was mixed together manually using a sterile spoon.

Note: To prevent and delay onset of fish oil oxidation, great care was taken when handling the fish oil emulsion and microparticle precursor composition containing the omega-3 fish oil. Nitrogen gas was used to create a gas blanket to reduce oxygen exposure during preparation of the microparticle precursor composition. The microparticle precursor composition was also mixed in a container purged with nitrogen gas to decrease exposure to air (oxygen, in particular) and covered with foil to decrease exposure of the fish oil to light.

### Example 5

### Microparticle Precursor Composition—Bifidobacterium lactis BB12

Preparing the Microparticle Precursor Composition—Bifidobacterium lactis BB12

### Materials:

Matrix Additive Composition of Example 1—130 g
Frozen Concentrate of Bifidobacterium lactis BB12—25 g
Fish Oil Emulsion of Example 2—500 mL
Cross-linkable Reagent of Example 3—400 g

### Method:

The frozen concentrate of Bifidobacterium lactis BB12 was melted in a sterile container at room temperature. Then, the microparticle precursor composition was prepared by combining very well all the sterile, prepared compositions listed below in a sterile container in following order:
1. Matrix Additive Composition of Example 1
2. Melted Concentrate of Bifidobacterium lactis BB12
3. Fish Oil Emulsion of Example 2
4. Cross-linkable Reagent of Example 3

The microparticle precursor composition was mixed together manually using a sterile spoon.

Note: To prevent and delay onset of fish oil oxidation, great care was taken when handling the fish oil emulsion and microparticle precursor composition containing the omega-3 fish oil. Nitrogen gas was used to create a gas blanket to reduce oxygen exposure during preparation of the microparticle precursor composition. The microparticle precursor composition was also mixed in a container purged with nitrogen gas to decrease exposure to air (oxygen, in particular) and covered with foil to decrease exposure of the fish oil to light.

### Example 6

### Microparticle Containing Lactobacillus casei Lc431

### Producing the Microparticle

### Materials:

Microparticle Precursor Composition of Example 4—˜1 L
Cross-linking Reagent: 0.1M sterile calcium chloride solution (autoclaved at 121° C.)—˜2 L

### Method:

The following method is in accordance with the method described in International Application No. PCT/AU2008/001695 (Publication No. WO 2009/062254).
1. A pressure tank was filled with the microparticle precursor composition. Another pressure tank was filled with the cross-linking reagent.
2. Compressed nitrogen gas supplies were connected via appropriate connections to the pressure tanks. The exit tubing on each tank was not connected initially.
3. The pressure gauges were adjusted to the pre-determined pressure shown in Table 1 below and the valves were locked. The bottom nozzle liquid is for the cross-linking reagent and the top nozzle liquid is for the microparticle precursor composition.

TABLE 1

| | Pressure (kPa) |
|---|---|
| Bottom nozzle nitrogen gas | 200 |
| Bottom nozzle liquid | 150 |
| Top nozzle nitrogen gas | 500 |
| Top nozzle liquid | 500 |

4. The liquid tubing of the cross-linking reagent pressure tank was connected to the reaction chamber and a cross-linking reagent mist was allowed to fill the reaction chamber for at least 2 minutes.
5. After 2 minutes, it was checked that the pressure in the microparticle precursor composition pressure tank is up to 500 kPa before connecting the liquid tubing of the pressure tank to the reaction chamber. An aerosol of the microparticle precursor composition was then produced and exposed to the cross-linking reagent mist.
6. The resulting microparticle slurry was collected from the collection tubing of the reaction chamber into a sterile container covered with foil.
7. After completion of the microparticle production, the pressure gauges were turned of and the apparatus was cleaned.
8. The microparticle slurry was filtered through a funnel layered with sterile Whatman filter paper (SC). The filtrate of wet microparticles was washed twice with sterile de-ionised water through the filter to wash out the calcium chloride residue.

### Example 7

### Microparticle Containing Bifidobacterium lactis BB12

### Producing the Microparticle

### Materials:

Microparticle Precursor Composition of Example 5—˜1 L
Cross-linking Reagent: 0.1M sterile calcium chloride solution (autoclaved at 121° C.)—˜2 L

### Method:

The following method is in accordance with the method described in International Application No. PCT/AU2008/001695 (Publication No. WO 2009/062254).
1. A pressure tank was filled with the microparticle precursor composition. Another pressure tank was filled with the cross-linking reagent.
2. Compressed nitrogen gas supplies were connected via appropriate connections to the pressure tanks. The exit tubing on each tank was not connected initially.
3. The pressure gauges were adjusted to the pre-determined pressure shown in Table 2 below and the valves were locked. The bottom nozzle liquid is for the cross-linking reagent and the top nozzle liquid is for the microparticle precursor composition.

TABLE 2

| | Pressure (kPa) |
|---|---|
| Bottom nozzle nitrogen gas | 200 |
| Bottom nozzle liquid | 150 |
| Top nozzle nitrogen gas | 500 |
| Top nozzle liquid | 500 |

4. The liquid tubing of the cross-linking reagent pressure tank was connected to the reaction chamber and a cross-linking reagent mist was allowed to fill the reaction chamber for at least 2 minutes.
5. After 2 minutes, it was checked that the pressure in the microparticle precursor composition pressure tank is up to 500 kPa before connecting the liquid tubing of the pressure tank to the reaction chamber. An aerosol of the microparticle precursor composition was then produced and exposed to the cross-linking reagent mist.
6. The resulting microparticle slurry was collected from the collection tubing of the reaction chamber into a sterile container covered with foil.
7. After completion of the microparticle production, the pressure gauges were turned of and the apparatus was cleaned.
8. The microparticle slurry was filtered through a funnel layered with sterile Whatman filter paper (5C). The filtrate of wet microparticles was washed twice with sterile de-ionised water through the filter to wash out the calcium chloride residue.

### Example 8

### Survival of Bifidobacterium lactis BB12 in the Microparticle

Microparticles prepared in accordance with Example 7 were added, while still wet, into milk for a study of Bifidobacterium lactis BB12 survival. The quantity of microparticles added to the milk was 0.5 g/100 mL and the milk was stored at 4° C. The probiotic loadings were measured as colony forming units per milliliter (CPU/mL).

The initial level of Bifidobacterium lactis BB12 was 8.46 log₁₀ CFU/mL. After 7 days, the level reduced to 8.40 log₁₀ CFU/mL, which corresponds to around 99% probiotic survival.

### Example 9

### Fish Oil Emulsion

### Preparing the Surfactant Mixture

### Materials:

Lecithin—10 g
De-ionised Water—90 g

### Method:

The surfactant mixture was prepared by dissolving lecithin in de-ionised water at ratio 1:9, using a mixer at a medium speed, until all lecithin was dissolved. The surfactant mixture was then sterilised at 90° C. for 30 minutes.

### Preparing the Fish Oil Emulsion

### Materials:

Surfactant Mixture as described above—100 g
Omega-3 Fish Oil—100 g
Water (Sterile)—200 g

### Method:

1, The primary fish oil emulsion was prepared by weighing the components into a sterile container. The mixture was then homogenised with a Silverson Heavy Duty Laboratory mixer/emulsifier at medium speed for 5 minutes. The mixer was washed in absolute alcohol and sterile water before use. No oil droplets were visible on the surface of the emulsion.
2. The primary fish oil emulsion was passed through a two-stage Twin Panda 400 (GEA Niro Soavi) homogeniser (First Stage: 250 bars, Second Stage: 50 bars) twice to further reduce the emulsion droplet size to produce the final fish oil emulsion. The homogenisation equipment was cleaned with disinfectant and sterile water before each use.
   Note: To prevent and delay onset of fish oil oxidation, great, care was taken when handling the fish oil and finished emulsion containing the omega-3 fish oil. Nitrogen gas was used to create a gas blanket to reduce oxygen exposure during preparation of the fish oil emulsion. The fish oil emulsion was also mixed in a container covered with foil to decrease light exposure.

### Example 10

### Cross-linkable Reagent

### Preparing the Cross-linkable Reagent

### Materials:

Sodium Alginate—20 g
Pectin—10 g
De-ionised Water—370 g

### Method:

The sodium alginate, pectin and de-ionised water were mixed together thoroughly. The cross-linkable reagent was then sterilised at 90° C. for 30 minutes the day before it was to be used.

### Example 11

### Microparticle Precursor Composition—Lactobacillus casei Lc431 and Fish Oil Containing Microparticles

Preparing the Microparticle Precursor Composition—Lactobacillus casei Lc431 and Fish Oil

### Materials:

Matrix Additive Composition of Example 1—75 g
Frozen Concentrate of Lactobacillus casei Lc431—25 g
Fish Oil Emulsion of Example 9—500 g
Cross-linkable Reagent of Example 10—400 g

### Method:

The frozen concentrate of Lactobacillus casei Lc431 was melted in a sterile container at room temperature. Then, the microparticle precursor composition was prepared by combining the sterile, prepared compositions listed below in a sterile container in following order:
1. Matrix Additive Composition of Example 1
2. Melted Concentrate of Lactobacillus casei Lc431
3. Fish Oil Emulsion of Example 0.9
4. Cross-linkable Reagent of Example 10

The microparticle precursor composition was mixed together manually using a sterile spoon.

Note: To prevent and delay onset of fish oil oxidation, great care was taken when handling the fish oil emulsion and microparticle precursor composition containing the omega-3 fish oil. Nitrogen gas was used to create a gas blanket to reduce oxygen exposure during preparation of the microparticle precursor composition. The microparticle precursor composition was also mixed in a container purged with nitrogen gas to decrease exposure to air (oxygen, in particular) and covered with foil to decrease exposure of the fish oil to light.

### Example 12

### Lactobacillus casei Lc431 and Fish Oil Containing Microparticles

### Producing the Microparticle

### Materials:

Microparticle Precursor Composition of Example 11—˜1 kg
Cross-linking Reagent: 0.1M sterile calcium chloride solution (autoclaved at 121° C. for 15 minutes and cooled to room temperature)—˜2 L

### Method;

The following method is in accordance with the method described in International Application No. PCT/AU2008/001695 (Publication No. WO 2009/062254).
1. A pressure tank was filled with the microparticle precursor composition. Another pressure tank was filled with the cross-linking reagent.
2. Compressed nitrogen gas supplies were connected via appropriate connections to the pressure tanks. The exit tubing on each tank was not connected initially.
3. The pressure gauges were adjusted to the pre-determined pressure shown in Table 3 below and the valves were locked. The bottom nozzle liquid is for the cross-linking reagent and the top nozzle liquid is for the microparticle precursor composition.

TABLE 3

| | Pressure (kPa) |
|---|---|
| Bottom nozzle nitrogen gas | 200 |
| Bottom nozzle liquid | 150 |
| Top nozzle nitrogen gas | 500 |
| Top nozzle liquid | 500 |

4. The liquid tubing of the cross-linking reagent pressure tank was connected to the reaction chamber and a cross-linking reagent mist was allowed to fill the reaction chamber for at least 2 minutes.
5. After 2 minutes, it was checked that the pressure in the microparticle precursor composition pressure tank is up to 500 kPa before connecting the liquid tubing of the pressure tank to the reaction chamber. An aerosol of the microparticle precursor composition was then produced and exposed to the cross-linking reagent mist.
6. The resulting microparticle slurry was collected from the collection tubing of the reaction chamber into a sterile container covered with foil.
7. After completion of the microparticle production, the pressure gauges were turned of and the apparatus was cleaned.
8. The microparticle slurry was filtered through a funnel layered with sterile Whatman filter paper (5C). The filtrate of wet microparticles was washed twice with sterile de-ionised water through the filter to wash out the calcium chloride residue.

### Example 13

### Liquid Sweet Formula Supplemented by Lactobacillus casei Lc431 and Fish Oil Containing Microparticles

Microparticles were prepared in accordance with Example 12. These microparticles were then coated with a coating composition comprising a blend of denatured whey protein isolate, canola oil, glycerol, trehalose, and water. The microparticles were coated by manually mixing together microparticles and the coating composition at a microparticle:coating composition ratio of 10:3 on a weight basis.

The microparticles were added to a liquid sweet formula to produce a supplemented formula comprising, on a weight basis: 0.45% xanthan gum, 1.8% carrageenan gum, 2% fructose, 34% mango syrup and 13% microparticles, with the remainder being water. Once supplemented with the microparticles, the liquid sweet formula was packaged to produce 10 mL serving pouches. Each pouch contained 3 billion CFU of Lactobacillus casei Lc431 and 100 mg DHA/EPA due to the supplementation by the microparticles.

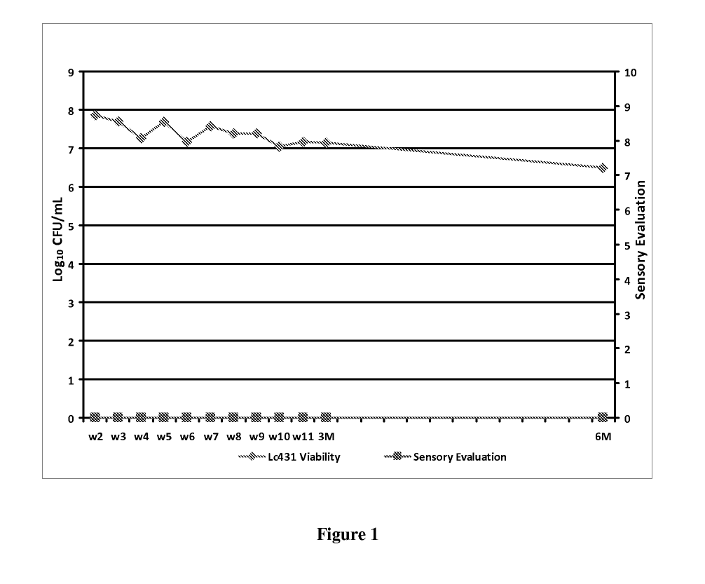

The pouches were stored initially at room temperature for two weeks and then at 4° C. The samples were tested over a six month period to assess probiotic survival and whether the flavour (i.e. smell/taste) of the Lactobacillus casei Lc431 and fish oil were perceptible. The results of these tests are shown below in Table 4 and in FIG. 1.

TABLE 4
Probiotic Viability and Flavour Perception Test Results.

| Measurement | Cell Viability | **Sensory |
|---|---|---|
| Date | (Log₁₀CFU/mL) | Evaluation |
| Week 2* | 7.85 | 0 |
| Week 3 | 7.7 | 0 |
| Week 4 | 7.26 | 0 |
| Week 5 | 7.68 | 0 |
| Week 6 | 7.17 | 0 |
| Week 7 | 7.58 | 0 |
| Week 8 | 7.38 | 0 |
| Week 9 | 7.38 | 0 |
| Week 10 | 7.05 | 0 |
| Week 11 | 7.16 | 0 |
| Week 12 | 7.15 | 0 |
| Month 6 | 6.50 | 0 |

NB:
*Stability test was started after 2 weeks storage at room temperature.
**Sensory evaluation rated from 0 = flavour (i.e. smell/taste) of the active(s) not detected to 10 = flavour of the active(s) detected very readily.

### Example 14

### Thin Base Drink Formula Supplemented by Lactobacillus casei Lc431 and Fish Oil Containing Microparticles

Microparticles were prepared in accordance with Example 12. These microparticles were then coated with a coating composition comprising a blend of denatured whey protein isolate, canola oil, glycerol, trehalose, and water. The microparticles were coated by manually mixing together microparticles and the coating composition at a microparticle:coating composition ratio of 10:3 on a weight basis.

The microparticles were added to a thin base drink formula to produce a supplemented formula comprising, on a weight basis: 3% Whey Protein Isolate, 2% Litess H from DuPont™ Danisco®, 1% Prebiotic Hi-Maize® from National Starch, 4% trehalose, 0.75% stevia, 0.05% xanthan gum, 0.1% potassium sorbate and 2% microparticles, with the remainder being water.

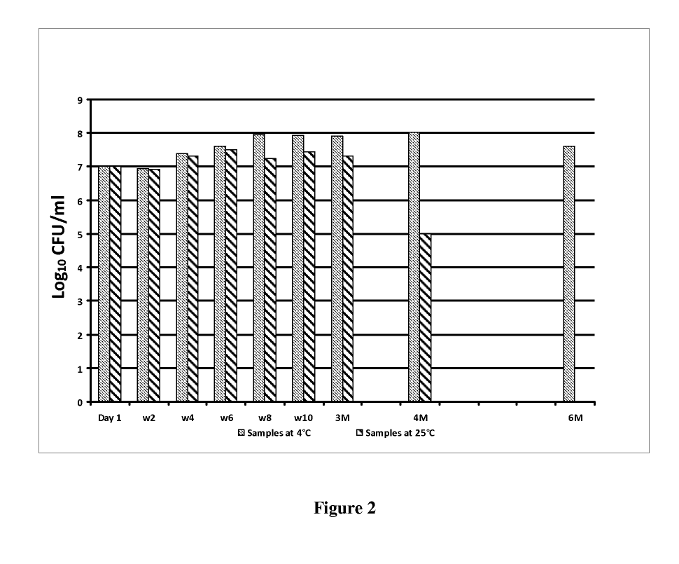

Samples of the supplemented thin base drink formula were stored at either 4° C. or 25° C. and tested over a six month period to assess probiotic survival. The results of these tests are shown below in Table 5 and FIG. 2.

TABLE 5
Probiotic Viability Test Results.

| | Cell Viability | Cell Viability |
|---|---|---|
| | for Samples | for Samples |
| Measurement | Stored at 4° C. | Stored at 25° C. |
| Date | (Log₁₀CFU/mL) | (Log₁₀CFU/mL) |
| Day 1 | 7.00 | 7.00 |
| Week 2 | 6.95 | 6.92 |
| Week 4 | 7.39 | 7.33 |
| Week 6 | 7.61 | 7.50 |
| Week 8 | 7.95 | 7.24 |
| Week 10 | 7.94 | 7.44 |
| Week 12 | 7.92 | 7.33 |
| Month 4 | 8.04 | 5.00 |
| Month 6 | 7.61 | |

### Example 15

### Survival of Lactobacillus casei Lc431 following storage—Lactobacillus casei Lc431 and Fish Oil Containing Microparticles

Microparticles were prepared in accordance with Example 12. These microparticles were then coated with a coating composition comprising a blend of denatured whey protein isolate, canola oil, glycerol, trehalose, and water. The microparticles were coated by manually mixing together microparticles and the coating composition at microparticle:coating composition ratio of 10:3 on a weight basis.

Samples of the microparticles were stored in a sealed container (i.e a container with a tight lid) at either 4° C. or −20° C. for a study of Lactobacillus casei Lc431 survival. The probiotic viability measurements taken from samples stored at 4° C. over a 3 month period are shown below in Table 6, while the probiotic viability measurements taken from samples stored at −20° C. over a four month period are shown below in Table 7.

TABLE 6
Probiotic Viability Test Results for Samples Stored at 4° C.

| | Batch 160812 | Batch 060912 | Batch 111112 |
|---|---|---|---|
| Measurement | Cell Viability | Cell viability | Cell viability |
| Date | (Log₁₀ CFU/g) | (Log₁₀ CFU/g) | (Log₁₀ CFU/g) |
| Day 0 | 9.75 | 9.92 | 9.95 |
| 1 month | 9.78 | 9.73 | n/a |
| 2 month | 9.74 | 9.78 | 9.48 |
| 3 month | 9.4 | 8.78 | 7.45 |

TABLE 7
Probiotic Viability Test Results for Samples Stored at −20° C.

| | Batch 160812 | Batch 060912 | Batch 111112 Cell |
|---|---|---|---|
| Measurement | Cell Viability | Cell viability | viability |
| Date | (Log₁₀ CFU/g) | (Log₁₀ CFU/g) | (Log₁₀ CFU/g) |
| Day 0 | 9.75 | 9.92 | 9.95 |
| 1 month | n/a | n/a | n/a |
| 2 month | 9.79 | 9.87 | 9.18 |
| 3 month | 9.62 | 9.62 | 9.18 |
| 4 month | 9.34 | 9.58 | n/a |

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Many modifications will be apparent to those skilled in the art without departing from the scope of the present invention.

1. A microparticle precursor composition comprising a blend of a probiotic, a cross-linkable reagent and an emulsion comprising a hydrophobic active.

2. A microparticle precursor composition according to claim 1, wherein the emulsion comprises the hydrophobic active, water and an emulsifier.

3. A microparticle precursor composition according to claim 1 or 2, wherein the cross-linkable reagent comprises sodium alginate.

4. A microparticle precursor composition according to claim 1, 2 or 3, wherein the cross-linkable reagent comprises pectin.

5. A microparticle precursor composition according to any one of claims 1 to 4, wherein the hydrophobic active is fish oil.

6. A microparticle precursor composition according to any one of claims 1 to 5, wherein the probiotic is selected from the group consisting of Lactobacillus casei Lc431, Lactobacillus rhamnosus CGMCC 1.3724, Bifidobacterium lactis BB12, Bifidobacterium lactis CNCM I-3446, Bifidobacterium longum ATCC BAA-999, Lactobacillus paracasei CNCM I-2116, Lactobacillus johnsonii CNCM I-1225, Lactobacillus fermentum VRI 003, Bifidobacterium longum CNCM I-2170, Bifidobacterium longum CNCM I-2618, Bifidobacterium breve, Lactobacillus paracasei CNCM I-1292, Lactobacillus rhamnosus ATCC 53103, Enterococcus faecium SF 68, Lactobacillus reuteri ATCC 55730, Lactobacillus reuteri ATCC PTA 6475, Lactobacillus reuteri ATCC PTA 4659, Lactobacillus reuteri ATCC PTA 5289, Lactobacillus reuteri DSM 17938, and mixtures thereof.

7. A method of producing a microparticle precursor composition comprising blending an emulsion comprising a hydrophobic active with a probiotic to form a probiotic-containing emulsion; and blending the probiotic-containing emulsion with a cross-linkable reagent.

8. A method according to claim 7, wherein each blending step comprises blending with a blending impeller operating at 300 rpm or less.

9. A method of producing microparticles comprising providing the microparticle precursor composition according to any one of claims 1 to 6 in a finely divided state;
and exposing the finely divided microparticle precursor composition to a cross-linking reagent that reacts with the cross-linkable reagent of the microparticle precursor composition to form microparticles.

10. A microparticle produced according to the method of claim 9 and comprising the hydrophobic active and the probiotic distributed within a cross-linked matrix.

11. A microparticle according to claim 10, wherein the probiotic is between 2.5% and 5% of the weight of the microparticle.

12. A microparticle according to claim 10 or 11, wherein the hydrophobic active is up to around 20% of the weight of the microparticle.

13. A product comprising microparticles according to claim 10, 11 or 12.
